(19) United States
(12) Patent Application Publication
(10) Pub. No.: US 2022/0177827 A1
(43) Pub. Date: Jun. 9, 2022

(54) BIOGAS PLANT AND BIOGAS TREATMENT

(71) Applicant: HITACHI ZOSEN INOVA AG, Zurich (CH)

(72) Inventors: Jens BECKER, Zurich (CH); Jan WIEGERS, Zurich (CH)

(73) Assignee: HITACHI ZOSEN INOVA AG, Zurich (CH)

(21) Appl. No.: 17/437,345

(22) PCT Filed: Mar. 6, 2020
(86) PCT No.: PCT/EP2020/056093

§ 371 Date: Sep. 8, 2021

(30) Foreign Application Priority Data
Mar. 8, 2019 (EP) 19161562.4

(51) Int. Cl. C12M 1/00; B01D 53/22; B01D 53/86; C12P 5/02; C12M 1/107; C10L 3/10

(52) U.S. Cl. CPC C12M 43/08; B01D 53/22; B01D 53/86; C12P 5/023; C12M 47/18; C10L 2290/46; C10L 3/104; C10L 2290/26; C10L 2290/548; C10L 2290/02; C10L 2290/06; C12M 21/04

(57) ABSTRACT

A biogas plant includes a fermenter, a biogas processing unit and a thermal engine. The biogas processing unit includes a gas separation unit and a compression unit for the separation of raw biogas from the fermenter into two gas streams, whereby the first gas stream includes a product gas that is enriched in biomethane with respect to the composition of the raw biogas and the second gas stream includes a residual gas that is enriched with carbon dioxide with respect to the composition of the raw biogas and has a biomethane concentration of less than 20%. The thermal engine produces energy that is used for operation of the compression unit.

The present invention relates to a biogas plant with a fermenter and a biogas treatment plant according to claim 1, and to a method for obtaining biomethane from raw biogas according to claim 15.

The term biogas usually refers to a mixture of mostly biomethane and carbon dioxide, which are formed through fermentation of organic substances in the absence of oxygen. Also the gas, which is liberated from landfills or digestion towers on sewage treatment plants may be called biogas. A biogas plant is therefore a plant for the production of biogas from organic substrates. Originally, biogas has been used in local cogeneration units to produce electric energy and heat, while parts of the generated heat could further be used for the biogas production process.

If biogas is needed at a location remote from its origin, processing of the biogas to biomethane and feeding thereof into a gas grid or a storage facility is necessary. For this purpose, biogas treatment plants have been developed, which are used for the separation of biogas into its components, mainly biomethane and carbon dioxide. The aim is to obtain an essentially pure biomethane gas stream, which is subsequently fed into a gas grid or can be used e.g. in the mobility industry.

Several methods for the purification of raw biogas to obtain biomethane have been successfully used in practice. Known methods include water scrubbing, organic scrubs (e.g. with amine-based solvents), pressure swing adsorption (PSA) and membrane based gas permeation methods.

Pressure swing adsorption methods include feeding of the biogas under elevated pressure into a solid-state bed reactor that is filled with an adsorbent. The so-called “heavy components” of the biogas mixture are adsorbed by the adsorbent and the so-called “light components” can be retrieved at the outlet of the bed in concentrated form. If the adsorption bed is saturated, part of the heavy components will also escape from the bed. As soon as this happens, the process is switched using valves, such that the outlet intended for the light components gets closed and a separate second outlet for the heavy components is opened. This switch comes along with a pressure reduction. At this reduced pressure the heavy absorbed components desorb from the bed and can be retrieved at the second outlet. Two interchangeably loaded and de-loaded adsorbents allow a continuous operation. If pressures below atmospheric pressure are used, the method is also termed VSA (Vacuum Swing Adsorption). If the pressure is partly above atmospheric pressure and partly below, then the method is termed VPSA (Vacuum Pressure Swing Adsorption). With the exception of the applied pressures and the necessary precautions associated therewith, all these methods are basically identical.

Membrane-based gas permeation methods include separation of carbon dioxide from the biogas with the aid of membranes. For that purpose, the raw biogas is pre-cleaned and compressed to pressures of usually 10 to 30 bar. At this pressure, carbon dioxide can easily permeate through a membrane that separates a permeate side from a retentate side. Other components of the raw biogas, in particular biomethane, cannot that easily permeate through the membrane, such that carbon dioxide (“residual gas”) can be collected on the permeate side (i.e. as “permeate”), while the desired cleaned biomethane is provided as “product gas” on the retentate side (i.e. as “retentate”), under the pressure of the original compression and ready for being fed into a gas grid. If multilayer membranes are used, they usually comprise a solvent-selective pore-free polymer film, a macroporous carrier membrane and a permeable fleece. Inorganic gas separation membranes e.g. based on zeolites, are available as alternative to polymer film based membranes. Irrespective of the membrane type multistep membrane systems are often used in order to obtain gas streams of biomethane and carbon dioxide with sufficiently high purity.

WO 2012/000727 A1 discloses a three-step process and an apparatus for separation of gas mixtures. The process and the apparatus consist of a feed stream separation stage and a retentate separation stage, of which both are membrane separation stages. The first retentate stream is heated to a temperature higher than the temperature of the feed stream, before it is introduced to the retentate separation stage, and the total capacity of the membranes used in the retentate separation stage is higher than the total capacity of the membranes used in the feed stream separation stage. The process and the apparatus are able to afford both permeate gas and retentate gases in relatively high purities with low re-circulation flow. This method is often used in practice and has been licensed to many suppliers in the past.

One problem of current biogas treatment methods is that they require a high amount of energy. For instance, the above-mentioned scrubbing methods require a lot of thermal energy, while the pressure swing adsorption methods and the membrane-based separation methods have a high energy demand for the compression of the raw biogas. Until now, the required compression energy has usually been exclusively provided by electric engines, which causes high production costs. Because of the continuously increasing demands with respect to purity of the biomethane and CO₂—and maximum tolerable amounts of pollutants therein—, multistep separation and cleaning systems are usually necessary. However, each cleaning step increases the overall energy consumption of the cleaning process.

Apart from the desired product gas of more or less pure biomethane, raw biogas also contains a variable amount of carbon dioxide that is obtained as a side product (residual gas). So far, this gas stream enriched in carbon dioxide was considered to be of little value and has usually been released into the environment. In view of the climate change, stricter regulations concerning emissions of gases containing greenhouse gases and/or pollutants have been established.

The problem to be solved by the present invention is therefore to provide a biogas plant comprising a biogas production facility for the production of biogas and a biogas treatment plant for the separation of raw biogas into biomethane and carbon dioxide, whereby the biogas plant has an improved energy and economical balance and at the same time meets the requirements with respect to purity of the gas streams obtained from the separation of the raw biogas.

This problem is solved by a biogas plant according to claim 1 and a method with the features of claim 15. Preferred embodiments are subject of the dependent claims.

A biogas plant in accordance with the present invention includes
a fermenter (or “digester”) for the production of raw biogas from organic biomaterial (or “biomass”), in particular organic waste,
a biogas treatment plant, and
a thermal engine.

The fermenter is capable of converting biomass into raw biogas and generally contains microorganisms that are capable of digesting the organic biomaterial. The fermenter can be an assigned technical unit (e.g. an apparatus) or a facility (e.g. a waste disposal site). The digestion—or “fermentation”—of the biomaterial leads to the production of raw biogas that contains biomethane.

To separate the biomethane in the raw biogas produced by the fermenter from other biogas components, in particular carbon dioxide (CO₂), the biogas treatment plant includes a gas separation unit that divides the raw biogas into at least a first and a second gas stream. The first gas stream consists of a product gas that is enriched in biomethane with respect to the composition of the raw biogas and the second gas stream consists of a residual gas that is enriched in carbon dioxide with respect to the composition of the raw biogas and has a biomethane concentration of less than 20%. The biogas treatment plant can comprise more than one separation step. Therefore the at least one residual gas stream can be a mixture of different intermediate residual gas streams gained from the different separation steps.

The separation of the gas streams can be achieved by using one of the known methods mentioned in the introduction part of this application, in particular by membrane separation or pressure swing adsorption. Since these separation methods generally require compression of the raw biogas to be separated, the biogas treatment plant includes a compression unit that can perform this task.

The thermal engine is part of the biogas plant and is provided with a combustion gas for combustion, which produces energy. The thermal engine can for example be a gas piston engine or a micro gas turbine. The thermal engine can be connected to an exhaust gas treatment unit. Said exhaust gas treatment unit is provided with an exhaust gas for treatment (usually for being cleaned). The thermal engine preferably also comprises at least one heat exchanger to utilize said energy.

A key aspect of the present invention is that energy produced by combustion of the combustion gas is used for operating the compression unit.

Thus, the inventive biogas plant allows that energy produced by the combustion of the combustion gas can be used for the operation of the biogas plant, specifically for operating the compression unit of the gas separation unit. This significantly reduces the operating costs of the biogas plant. Heat energy produced by the thermal engine may also be transferred to the fermenter, which reduces the operating costs of the biogas plant.

The thermal engine and the compression unit are generally mechanically connected, preferably via a rigid coupling.

The energy transfer from the thermal engine to the compression unit can be either direct (e.g. via a rigid or variable coupling) or indirect, e.g. with an energy converter or storage in between. In consequence, operation of the compression unit requires less externally produced energy (i.e. electric energy or fuel produced outside of the biogas plant), which can reduce the overall biomethane production costs.

In a particularly preferred embodiment of the invention, at least a fraction of the residual gas is fed to the thermal engine as part of the combustion gas and/or to the gas treatment unit as part of the exhaust gas.

In other words, preferably, at least a fraction of the residual gas (obtained from the separation of the raw biogas within the gas separation unit) is delivered to one or both of the thermal engine and the exhaust gas treatment unit. Of the residual gas fed to the thermal engine and/or the exhaust gas treatment unit, a fraction X (with X being between 0% and 100%) is delivered as part of the combustion gas and another fraction 100%−X may be delivered to the exhaust gas treatment unit as part of the exhaust gas.

Preferably, the exhaust gas treated by the exhaust gas treatment unit includes exhaust gases from the combustion process within the thermal engine. This way, the exhaust gases produced during combustion of the combustion gas can be cleaned, in particular by removal of incompletely burned carbon compounds, such as e.g. NMHC, carbon monoxides or methane, and/or nitrous oxides. In this embodiment, any residual gas delivered to the exhaust gas treatment unit is preferably mixed with the exhaust gases from the thermal engine before being fed into the exhaust gas treatment unit.

In the above-mentioned preferred embodiment, the biogas plant according to the present invention allows for an improved environmental balance, since any biomethane residue present in the residual gas can be cleaned in the exhaust gas treatment plant and/or converted into carbon dioxide and water during its combustion in the thermal engine and is therefore not released as a potent greenhouse gas into the environment.

Generally, the goal of a gas separation unit is to minimize the amount of biomethane within the residual gas, i.e. the methane slip, to avoid release of biomethane—which is a greenhouse gas—into the atmosphere. A reduction in methane slip usually goes along with a more complex construction of the gas separation unit (e.g. increased number of separation stages, larger membrane surface area etc.). However, since the biogas plant according to the present invention allows for combustion of residual biomethane within the residual gas for energy production (and/or at least allows for removing it with the aid of the exhaust gas treatment unit, e.g. by means of oxidation), the gas separation unit of the inventive biogas plant could also be significantly simplified, without risking increased emissions of biomethane being released into the atmosphere.

In summary, thanks to reduced need for external energy and significant reduction of the amount of biomethane that is liberated into the atmosphere, the biogas plant in accordance with the present invention provides a particularly energy-efficient, economically and environmentally friendly production of biomethane. If a simplified separation unit is used, the building and maintenance costs as well as the operating costs of the biogas plant can be further reduced.

Preferably, any residual gas that is not fed to the thermal engine is cleaned in the exhaust gas treatment unit, before being released into the atmosphere.

In a preferred embodiment of the biogas plant, the combustion gas includes a variable amount of natural gas and/or raw biogas from the fermenter and/or product gas. This means that in this preferred embodiment, the combustion gas may theoretically consist of only residual gas, yet it will generally additionally include fractions of the named combustible gases (e.g., natural gas, raw biogas or biomethane). The addition of combustible gases to the combustion gas is usually required, because depending on the separation efficiency of the gas separation unit (i.e. the methane slip of the gas separation unit)—the residual fraction of biomethane in the residual gas may be very small, such that the latter may not contain sufficient combustible components any more to allow adequate combustion within the thermal engine for energy production. In this case, a variable amount of natural gas and/or raw biogas and/or product gas, preferably raw biogas, can be added to the combustion gas to drive the combustion process. If applicable, also other combustibles, e.g. LPG (propane/butane), can be added to the combustion gas. This ensures that also in case of low biomethane concentrations in the residual gas, a constant operation of the thermal engine and therefore a constant energy production can be guaranteed. The minimal concentration of combustible gases within the combustion gas is determined by the type of thermal engine used. Pilot injection engines are one example of engines that allow combustion of lean gases, i.e. gases with high concentrations of non-combustible gases, such as carbon dioxide.

In case that the thermal engine produces energy that cannot be utilized by the compression unit at a certain point in time, the extra energy is preferably fed to and utilized by other units of the biogas plant, e.g. the fermenter. Alternatively, such extra energy may also be fed into an energy storage or local energy distribution grid.

Preferably, the residual gas includes a biomethane concentration of less than 15%, preferably less than 10%, more preferably less than 5%, more preferably within the range of 0.1% to 3% or even within the range of 0.1% to 1%.

Preferably, the combustion gas fed to the thermal engine comprises exclusively gases of the biogas plant itself, e.g. product gas from the separation unit, residual gas from the separation unit and/or raw biogas from the fermenter. This means that in this embodiment, no external combustion gas (e.g. from a local natural gas grid) is required and the biogas plant can therefore be operated autonomously and location-independent. If no gas grid connection is present, the produced biomethane can be used immediately or filled into containers and transported in liquid or gaseous form, either to a consumer or to a facility, where it can be fed into a natural gas grid.

The exhaust gas treatment unit of the biogas plant is preferably a catalytic gas processing unit and can be used for cleaning of residual gas or other exhaust gases. Specifically, it is preferably also used to clean the exhaust gases from the thermal engine before releasing them into the atmosphere. Methods for removing harmful substances, in particular nitrogen oxides, unburnt hydrocarbons and carbon monoxides are known to a person skilled in the art. For the removal of hydrocarbons and carbon monoxides common oxidation catalysts are often used (See e.g. J. K. Lampert et al, Applied Catalysis B: Environmental, Vol. 14, Issues 3-4, Dec. 29, 1997, Pages 211-223).

The gas separation unit of the biogas plant is preferably a membrane separation plant and/or a pressure swing adsorption plant. In comparison to other methods, gas separation by means of a membrane separation plant or a pressure swing absorption plant is more energy-efficient and cost saving.

As mentioned, one advantage of the inventive biogas plant is that it can include a gas separation unit of simplified construction. “Simplified” means that in comparison to the common separation units, e.g. common membrane separation plants, the inventive biogas plant can have a reduced number of separation steps, a reduced number of membrane modules and/or an overall reduced size of membrane area. This reduction in separation steps or number or size of membrane module comes along with a higher methane slip. But as described above, the amount of residual biomethane within the residual gas can be utilized by the thermal engine for energy production. Nevertheless, it is generally preferred that the biomethane concentration in the residual gas is below 15%, preferably below 10%, more preferably below 5%.

In one embodiment, the separation unit is preferably a simplified membrane separation plant with two separation steps at most, particularly preferably with only one separation step. Compared to units having more than two separation steps, the inventive membrane separation unit requires substantially less energy, since each additional separation step increases the energy demand of the separation unit. In addition, the construction of a gas separation unit having only two or fewer separation steps is usually simpler and therefore cost saving and less demanding with respect to maintenance.

If a common gas (piston) motor is used as thermal engine, the combustion gas generally contains about 40% to 50% combustible material, usually biomethane. As mentioned, special lean gas motors are available, which are usually more expensive but can work with much lower methane concentrations in the combustion gas. From an economic point of view, the combustion gas preferably includes a biomethane concentration of at least 10%, more preferably at least 20%, even more preferably at least 30%.

Irrespective of the biomethane concentration within the residual gas in one embodiment it is preferred that all residual gas obtained in the gas separation unit is delivered directly or indirectly (e.g. via the fermenter or intermediate storage) to the thermal engine and/or exhaust gas treatment unit, preferably without prior treatment (treatment hereby refers to processes that change the composition of the residual gas).

In one embodiment, all residual gas is fed to the thermal engine. Direct utilization of the residual gas saves costs and is thus particularly advantageous from an economic point of view.

In accordance with a preferred embodiment, the biogas plant includes two gas pipelines, one of which serves for the delivery of raw biogas from the fermenter to the biogas treatment plant and the other serves for the delivery of raw biogas from the fermenter to the thermal engine. The two pipelines may be entirely separated or have a common origin that later divides into two separate pipelines. The provision of two pipelines allows that delivery of raw biogas to the gas separation unit and/or to the thermal engine can occur in dependence of the occupancy rate of the respective consumption unit (consumption unit refers in this respect to the thermal engine and the gas separation unit). For instance, all raw biogas from the fermenter can be fed to only one of the consumption units. Alternatively, the raw biogas may also be divided into equal or unequal parts between the two consumption units. The amount of raw biogas delivered to a certain consumption unit is preferably regulated by means of valves, whose through-put is regulated with the aid of a control unit in dependence of the energy requirement of the pertaining consumption unit at the certain point in time.

In view of a particularly energy-efficient operation of the biogas plant, at least part of the thermal energy that is generated during compression of the raw biogas and/or by the thermal engine is preferably used for operating the fermenter. As a result, energy that would otherwise be necessary for heating the fermenter can be saved. The thermal energy generated from the compression unit is preferably distributed within the fermenter. If the thermal energy from the gas compression and the thermal engine is not sufficient for the fermenter's operation, additional external thermal energy may be provided to the fermenter. On the other hand, should more thermal energy be produced than required by the fermenter, the extra energy is preferably fed into a heat storage or a thermal energy grid or is used otherwise within or nearby the biogas plant.

In a further aspect the present invention also relates to a method for the production of biomethane from raw biogas, whereby the method comprises at least the following steps:

Firstly, raw biogas produced by a fermenter is compressed with the aid of a compression unit. The compressed raw biogas is then separated with the aid of a gas separation unit into a product gas, which is enriched in biomethane with respect to the composition of the raw biogas, and into a residual gas that is enriched in carbon dioxide with respect to the composition of the raw biogas. The residual gas comprises less than 20% biomethane.

In accordance with the present invention, the combustion of a combustion gas within a thermal engine produces energy and at least part of said energy is used for operation of the compression unit.

Preferably, the method comprises further a step in which at least a fraction of the residual gas is delivered as part of the combustion gas to the thermal engine and/or as part of an exhaust gas to an exhaust gas treatment unit.

Preferably, the thermal engine comprises at least one heat exchanger for utilizing the thermal energy that is produced by the thermal engine.

This reutilization of energy in accordance with the inventive operation method allows that the amount of external energy (e.g. electricity or gas from outside of the biogas plant) used for the operation of the biogas plant can be reduced, preferably to zero. The production of biomethane takes preferably place within a biogas plant as presented above. Thus, the separation of the raw biogas takes preferably place within a gas separation unit that comprises a membrane separation plant and/or a pressure swing adsorption plant. As described above in connection with the preferred embodiments of the inventive biogas plant, gas separation with the aid of a membrane separation unit or a PSA-unit is more energy-efficient and cost saving compared to other known gas separation methods.

In view of an improved energy balance, some energy produced by the thermal engine (electricity or thermal energy) and/or thermal energy generated in the compression unit may also be used for the operation of the fermenter.

It is preferred that the thermal engine receives a variable amount of raw biogas from the fermenter and/or of product gas from the separation unit, depending on the biomethane concentration of the residual gas. In this case, energy can be produced within the thermal engine without utilizing externally produced energy or fuel (e.g. natural gas) and this allows to use the inventive method in locations that are not connected to a common electricity or gas grid.

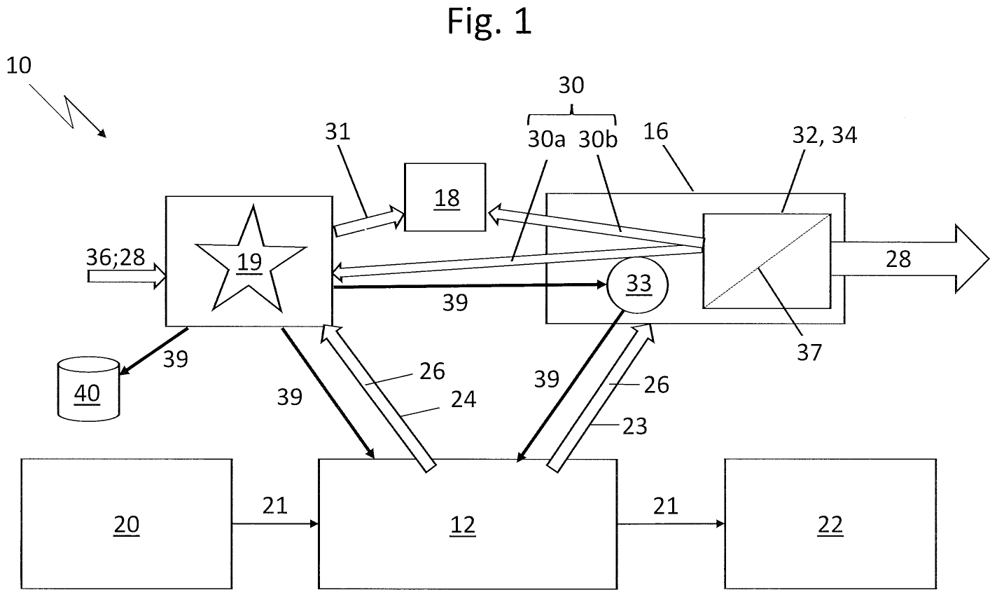

In the following the present invention will be further explained with reference to the embodiment presented in FIG. 1, which shows

FIG. 1 a schematic representation of a biogas plant of the present invention.

An embodiment of a biogas plant 10 in accordance with the present invention as shown in FIG. 1 includes a fermenter 12, a thermal engine 19 and a biogas treatment plant 16 for the production of biomethane 17. The thermal engine 19 is provided with a combustion gas. The biogas plant 10 further includes an exhaust gas treatment unit 18 that is provided with an exhaust gas. The thermal engine 19 and the exhaust gas treatment unit 18 are preferably connected, such that exhaust gases from the thermal engine 19 can be delivered to the exhaust gas treatment unit 18.

The term “thermal engine” thereby generally refers to a combustion engine, e.g. a gas (piston) engine, a micro gas turbine or a pilot injection engine, which uses a combustion gas—e.g. natural gas, biogas, or hydrogen—instead of or in addition to a liquid fuel for the conversion of chemical energy into mechanical and/or thermal energy. The thermal engine generally also includes at least one heat exchanger (not shown).

In case of the biogas plant 10 of FIG. 1, a substrate preparation unit 20 provides the fermenter 12 with organic material (biomass) 21. The biomass 21 is fermented in the fermenter 12 and subsequently transported to a substrate postprocessing unit 22.

During the fermentation of the biomass 21 within the fermenter 12, raw biogas 26 is produced. In the shown embodiment, raw biogas 26 from the fermenter 12 is delivered to the biogas treatment plant 16 through a first gas pipeline 23. A second gas pipeline 24 is provided for the delivery of raw biogas 26 from the fermenter 12 to the thermal engine 19. The two pipelines 23, 24 may be entirely separated or have a common origin that later divides into two separate pipelines. The fraction of raw biogas 26 fed to the thermal engine 19 is generally smaller than the fraction that is fed to the biogas treatment plant 16.

Within the biogas treatment plant 16 the raw biogas 26 is separated into a product gas 28 and a residual gas 30. The separation is performed with the aid of a gas separation unit 32 and a compression unit 33 for compression of the raw biogas 26 to be separated. The gas separation unit 32 comprises a membrane separation plant 34.

The membrane separation plant 34 includes at least one membrane 37, by means of which the raw biogas 26 is separated into a first gas stream comprising the product gas 28 and a second gas stream comprising the residual gas 30. The separation occurs on grounds of different permeabilities of biomethane and carbon dioxide through the membrane. The product gas 28 comprises a higher biomethane concentration with respect to the composition of the raw biogas 26, whereas the residual gas 30 comprises more carbon dioxide than the composition of the raw biogas 26. The residual gas 30 contains less than 20% biomethane.

The gas separation unit 32 may be simplified in its construction, which means that it involves less separation steps and/or a reduced membrane area compared to gas separation units known in the art that are built for minimizing the amount of residual biomethane within the residual gas, i.e. the methane slip. Since the biogas plant 10 according to the present invention allows for using the residual biomethane within the residual gas 30 for energy production (see next paragraph), the gas separation unit 32 of the inventive biogas plant may be significantly simplified, if desired.

In the shown embodiment of the biogas plant 10, some of the residual gas 30 from the separation unit 32 is fed to the thermal engine 19 and/or the exhaust gas treatment unit 18. Thus, a fraction 30a between 0% and 100% of said residual gas 30 is fed to the thermal engine 19 and forms part of the combustion gas that is combusted within the thermal engine 19. In the shown embodiment presented in FIG. 1, a remaining fraction 30b of the residual gas 30 is fed to the exhaust gas treatment unit 18 and forms part of the exhaust gas to be treated (cleaned) in the exhaust gas treatment unit 18. The exhaust gas to be cleaned by means of the gas treatment unit 18 may further comprise exhaust gases 31 from the thermal engine 19. Thus, in the shown embodiment, the exhaust gas treatment unit 18, e.g. a catalytic gas processing unit, serves for cleaning i) the fraction 30b of residual gas 30 that is not fed to the thermal engine 19 and ii) exhaust gases 31 from the thermal engine 19, such that the latter can be cleaned, in particular from incompletely burnt carbon compounds (e.g. NMHC, carbon monoxides or methane) and/or nitrogen oxides, before releasing it into the atmosphere.

By combustion of the combustion gas within the thermal engine 19, mechanical and/or thermal energy 39 is produced. Said energy 39 is used for the operation of the biogas treatment plant 16, specifically the compression unit 33. It may additionally be used for other purposes, e.g. for operation of the fermenter 12, or it may be stored in a heat storage 40. If the content of combustible material (in particular biomethane) in the residual gas 30 is too low to allow adequate combustion and thus energy production within the thermal engine 19, additional other combustibles—e.g. raw biogas 26 from the fermenter 12 or natural gas 36 from an external source (e.g. from a public gas grid (not shown)) or product gas 28 from the biogas treatment plant 16 can be added to the combustion gas of the thermal engine 19. In addition, at least part of thermal energy 39 produced by the thermal engine 19 and/or the compression unit 33 can also be used for heating the fermenter 12.

1. Biogas plant including
a fermenter for the production of raw biogas from biomass,
a thermal engine, which is provided with a combustion gas for combustion for the production of mechanical and/or thermal energy,
a biogas treatment plant including a compression unit and a gas separation unit for the separation of the raw biogas into a first and second gas stream,
wherein the first gas stream consists of a product gas that is enriched in biomethane with respect to the composition of the raw biogas, and the second gas stream consists of a residual gas that comprises less than 20% biomethane and is enriched in carbon dioxide with respect to the composition of the raw biogas,
and an exhaust gas treatment unit, which is provided with an exhaust gas for treatment,
wherein at least part of the energy produced by combustion of the combustion gas in the thermal engine is used for operating the compression unit.

2. Biogas plant according to claim 1, wherein at least one of the combustion gas and the exhaust gas comprises at least a fraction of the residual gas.

3. Biogas plant according to claim 1, wherein the residual gas has a biomethane concentration of less than 15%.

4. Biogas plant according to claim 1, wherein the combustion gas includes a variable amount of natural gas and/or raw biogas of the fermenter and/or product gas.

5. Biogas plant according to claim 1, wherein the exhaust gas includes exhaust gases from the thermal engine.

6. Biogas plant according to claim 1, wherein at least one of the combustion gas and the exhaust gas consists of gases from the biogas plant.

7. Biogas plant according to claim 1, wherein the exhaust gas treatment unit is a catalytic gas processing unit.

8. Biogas plant according to claim 1, wherein the thermal engine and the compression unit are mechanically connected via a rigid coupling.

9. Biogas plant according to claim 1, wherein the gas separation unit includes a simplified membrane separation plant.

10. Biogas plant according to claim 1, wherein the gas separation unit has a methane slip of within the range of 0.1% to 5%.

11. Biogas plant according to claim 1, wherein the combustion gas includes a biomethane concentration of at least 10%.

12. Biogas plant according to claim 1, wherein all residual gas from the gas separation unit is delivered directly or indirectly to the thermal engine and/or the exhaust gas treatment unit.

13. Biogas plant according to claim 1, characterised by a first gas pipeline for the delivery of raw biogas from the fermenter to the biogas treatment plant and a second gas pipeline for the delivery of raw biogas from the fermenter to the thermal engine.

14. Biogas plant according to claim 1, wherein thermal energy produced during the compression of the raw biogas and/or by the thermal engine is used for operating the fermenter.

15. Method for obtaining biomethane from raw biogas according to claim 1, the method comprising at least the following steps:
a) raw biogas from a fermenter is compressed with the aid of a compression unit;
b) the compressed raw biogas is separated with the aid of a gas separation unit into a product gas that is enriched in biomethane in relation to the raw biogas and into a residual gas that has a biomethane concentration of less than 20% and is enriched in carbon dioxide in relation to the raw biogas; and
c) combustion of the combustion gas in a thermal engine produces energy that is at least partly used for operating the compression unit.

16. Method according to claim 15, wherein at least a fraction of the residual gas is delivered as part of a combustion gas to the thermal engine and/or as part of an exhaust gas to an exhaust gas treatment unit.

17. Method according to claim 15, wherein at least part of the energy produced within the thermal engine and/or the compression unit is used for operating the fermenter.
